(19) United States
(12) Patent Application Publication
(10) Pub. No.: US 2026/0144650 A1
(43) Pub. Date: May 28, 2026

(54) GEAR SET

(71) Applicant: The Regents of the University of Michigan, Ann Arbor, MI (US)

(72) Inventor: Revanth Damerla, Ann Arbor, MI (US)

(21) Appl. No.: 19/120,913

(22) PCT Filed: Oct. 16, 2023
(86) PCT No.: PCT/US2023/035176

§ 371 Date: Apr. 14, 2025

Related U.S. Application Data
(60) Provisional application No. 63/416,649, filed on Oct. 17, 2022.

(51) Int. Cl. A61F 2/58; A61F 2/68; A61F 2/70

(52) U.S. Cl. CPC A61F 2/586; A61F 2/70; A61F 2002/6836

(57) ABSTRACT

A gear set comprising an input shaft, an output shaft, a gear; and a friction pad. In a first configuration, the gear set has a first gear ratio for the input shaft rotational speed to the output shaft rotational speed equal to N:1, where N is not equal to 1. In the first configuration, the gear rotates relative to the friction pad. In a second configuration, the gear set has a gear ratio for the input shaft rotational speed to the output shaft rotational speed equal to 1:1, wherein the friction pad prevents rotation of the gear relative to the friction pad.

### RELATED APPLICATIONS

This application claims priority to and the benefit of U.S. Provisional Patent Application Ser. No. 63/416,649 filed on Oct. 17, 2022, which is incorporated herein by reference in its entirety for all purposes.

### GOVERNMENT FUNDING

This invention was made with Government support under Federal Grant No. DGE 1256260 awarded by the National Science Foundation. The Federal Government has certain rights to the invention.

### BACKGROUND

An upper limb prosthetic is an artificial device that serves as a substitute for a partially or entirely lost hand or arm due to an accident, injury, illness, trauma, or congenital defect. The goal of any prosthesis is to provide the user with the ability to perform activities of daily living to regain independence. However, existing designs do not meet all user needs, including, but not limited to, total weight, dexterity, speed, strength, and functionality.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides an assembly comprising: an input shaft; a sun gear coupled to the input shaft for co-rotation with the input shaft; a ring gear with an inner surface and an outer surface; a carrier; and a planet gear coupled to the carrier and enmeshed with the inner surface of the ring gear. The assembly further includes an output shaft coupled to the carrier for co-rotation with the carrier and a friction pad positioned between the planet gear and the carrier, or positioned between the carrier and the ring gear, or positioned between the planet gear and the ring gear. The assembly further includes a clutch engageable with the outer surface of the ring gear; wherein the assembly is movable between a first configuration and a second configuration. In the first configuration, the clutch engages the outer surface of the ring gear and the assembly has a first gear ratio of the input shaft rotational speed to the output shaft rotational speed equal to N:1, where N is greater than 0 and not equal to 1. In the second configuration, the clutch disengages the outer surface of the ring gear and the assembly has a second gear ratio of the input shaft rotational speed to the output shaft rotational speed equal to 1:1.

In some embodiments, when in the second configuration, the ring gear co-rotates with the output shaft.

In some embodiments, the friction pad is made of rubber, foam, or graphite.

In some embodiments, when in the second configuration, the friction pad prevents the planet gear from rotating about a planet gear axis.

In some embodiments, when in the first configuration, the planet gear rotates about the planet gear axis relative to the friction pad.

In some embodiments, the ring gear includes a first portion and a second portion coupled to the first portion, wherein a cavity is at least partially defined by first portion and the second portion.

In some embodiments, the planet gear, the friction pad, and the carrier are positioned within the cavity.

In some embodiments, the clutch includes a linear actuator.

In some embodiments, the clutch includes an electrostatic clutch.

One aspect of the present disclosure provides a gear set comprising: an input shaft; an output shaft; a gear; and a friction pad. In a first configuration the gear set has a first gear ratio for the input shaft rotational speed to the output shaft rotational speed equal to N:1, where N is not equal to 1, and wherein the gear rotates relative to the friction pad in the first configuration. In a second configuration the gear set has a gear ratio for the input shaft rotational speed to the output shaft rotational speed equal to 1:1, and wherein the friction pad prevents rotation of the gear relative to the friction pad.

In some embodiments, the gear set is a cycloidal gear set.

In some embodiments, the gear set is a Ravigneaux gear set.

In some embodiments, the gear set is a planetary gear set.

In some embodiments, the gear is a planet gear.

In some embodiments, the gear is a ring gear.

One aspect of the present disclosure provides a gear set comprising: an input shaft; an output shaft; a plurality of gears including at least one frictional feature. The at least one frictional feature causes the plurality of gears to rotate in unison when an input torque on the input shaft is below a threshold torque. The gear set has a gear ratio for the input shaft rotational speed to the output shaft rotational speed equal to 1:1 when the input torque is below the threshold torque.

In some embodiments, the at least one frictional feature is a friction pad.

In some embodiment, the at least one frictional feature is an interference fit, a protrusion, a friction pad, or a combination thereof.

In some embodiments, the plurality of gears are part of a planetary gear set, a cycloidal gear set, or a Ravigneaux gear set.

One aspect of the present disclosure provides an assembly comprising: an electric motor; and a gear set including a friction pad. The gear set is coupled to the electric motor. The assembly further includes a linkage coupled to the gear set, and a prosthetic coupled to the linkage. In a first configuration the gear set has a first gear ratio of N:1, where N is not equal to 1. In a second configuration the gear set has a gear ratio of 1:1.

In some embodiments, the linkage includes a ball screw, a lead screw, a cable, a four-bar linkage, or a six-bar linkage.

In some embodiments, the prosthetic is a finger with a finger joint and the link is coupled between the ball screw and the finger joint.

In some embodiments, the prosthetic is a finger, a thumb, a wrist, a forearm, a shoulder, a leg, a bird wing, a snake body, or a fish tail.

In some embodiments, the two-speed gear set is a first two-speed gear set and the assembly further includes a second two-speed gear set coupled to the first two-speed gear set.

Other aspects of the disclosure will become apparent by consideration of the detailed description and accompanying drawings.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (also “FIG.”) relating to one or more embodiments.

FIG. 1 is a schematic of a motor, a transmission, and a prosthetic.

FIG. 2 is a perspective view of a motor, a transmission, and a prosthetic (e.g., a finger).

FIG. 3 is a perspective view of FIG. 2 with portions removed for clarity.

FIG. 4 is a cross-sectional view of FIG. 2.

FIG. 5 is an enlarged portion of FIG. 4.

FIG. 6 is perspective view of a portion of a gear set.

FIG. 7 is a side perspective view of a gear set.

FIG. 8 is a disassembled view of the gear set of FIG. 7.

FIG. 9 illustrates partially disassembled view of the gear set of FIG. 7.

FIG. 10 is a graph of torque versus speed for a motor and a gear set disclosed herein, illustrating a first operating mode and a second operating mode (e.g., a direct drive move).

FIG. 11 is a perspective view of a perspective view of an assembly including a motor, a gear set, and a linkage.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” and “approximately” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations when interpreted in the alternative (“or”).

Moreover, the present disclosure also contemplates that in some embodiments, any feature or combination of features set forth herein can be excluded or omitted. To illustrate, if the specification states that an apparatus comprises components A, B, and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

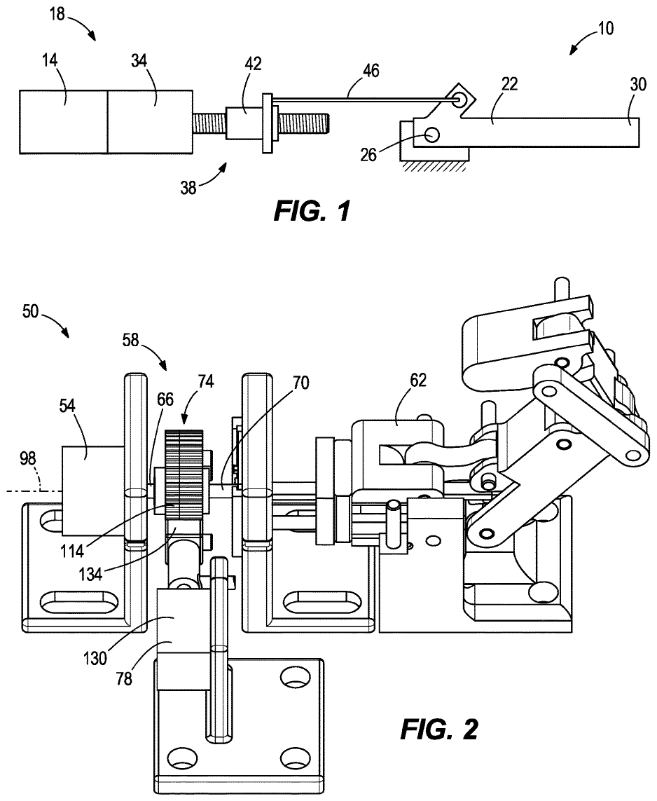

With reference to FIG. 1, a schematic of a prosthetic assembly 10 is illustrated with a motor 14 (e.g., a rotary actuator), a transmission 18, and a prosthetic 22. In the illustrated embodiment, the prosthetic 22 includes a finger with a finger joint 26 and a phalanx 30. In other embodiments, the prosthetic 22 is a finger, a thumb, a hand, a wrist, a forearm, a shoulder, a leg, a humanoid robot, a robotic bird wing, a robotic fish tail, a multi-legged robot (e.g., a dog, a spider), other non-bioinspired robotics, robot-assisted medical devices, or any other suitable prosthetic limb.

In the illustrated embodiment, the transmission 18 includes a gear set 34 (e.g., a 2-speed active gearbox), and a linkage 38 coupled to the gear set 34. In the illustrated embodiment, the linkage 38 includes a ball screw 42 and a cable 46 (e.g., a link). In other embodiments, the linkage 38 includes a ball screw, a lead screw, a cable, a four-bar linkage, a six-bar linkage, a multi-bar linkage, an additional gear set, any suitable transmission component, or any combination thereof. The transmission 18 couples the motor 14 output to the finger joint 26 and the phalanx 30. In the illustrated embodiment, the gear set 34 is coupled to the output shaft of the motor 14. In some embodiments, the ball screw 42 is coupled to the gear set 34, and the cable 46 is coupled between the ball screw 42 and the prosthetic 22. In the illustrated embodiment, the cable 46 is coupled between the ball screw 42 and the finger joint 26. As detailed further herein, the gear set 34 includes at least one friction pad (e.g., at least one frictional feature) and is movable between a first configuration where the gear set 34 has a first gear ratio of N:1, where N is not equal to 1, and a second configuration where the gear set 34 has a gear ratio of 1:1.

With reference to FIG. 2, a prosthetic assembly 50 is illustrated with an electric motor 54 (e.g., a brushless DC motor), a transmission assembly 58 (e.g., a transmission), and a prosthetic 62 (e.g., a finger).

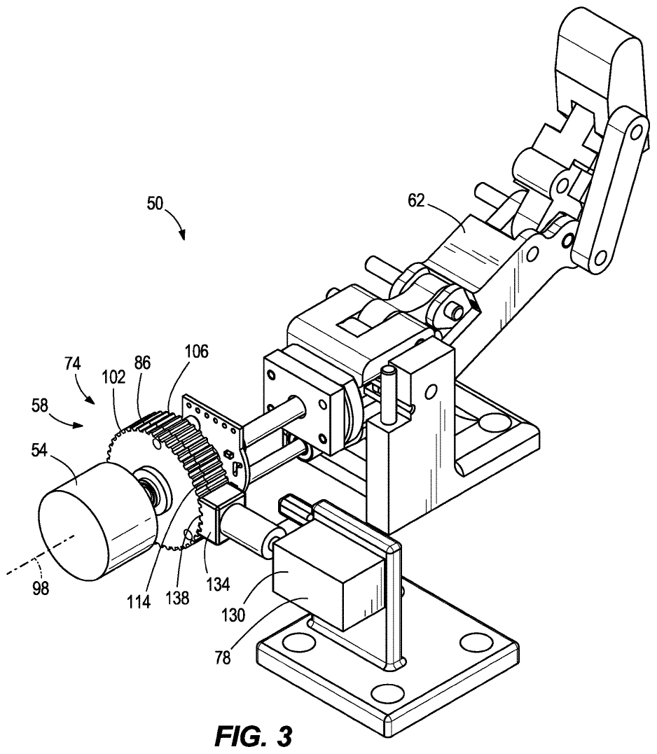

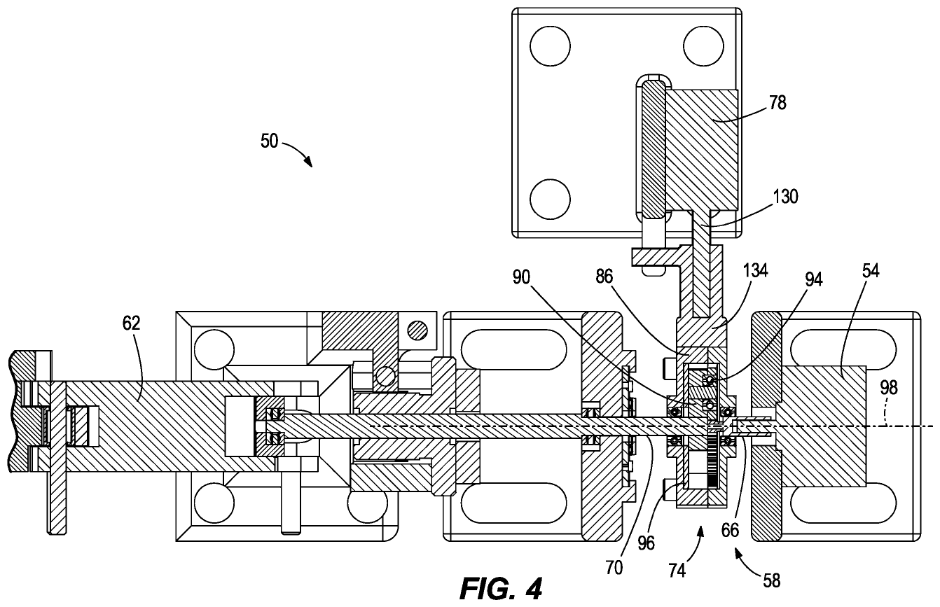

With reference to FIGS. 3 and 4, the transmission assembly 58 includes an input shaft 66, an output shaft 70, a gear set 74, and a clutch 78. In the illustrated embodiment, the gear set 74 is a planetary gear set. In other embodiments, the gear set 74 is a cycloidal gear set, Ravigneaux gear set, a harmonic gear set, or other suitable gear set. In other words, aspects of the present disclosure are used in various gear sets and is not limited to planetary gear sets.

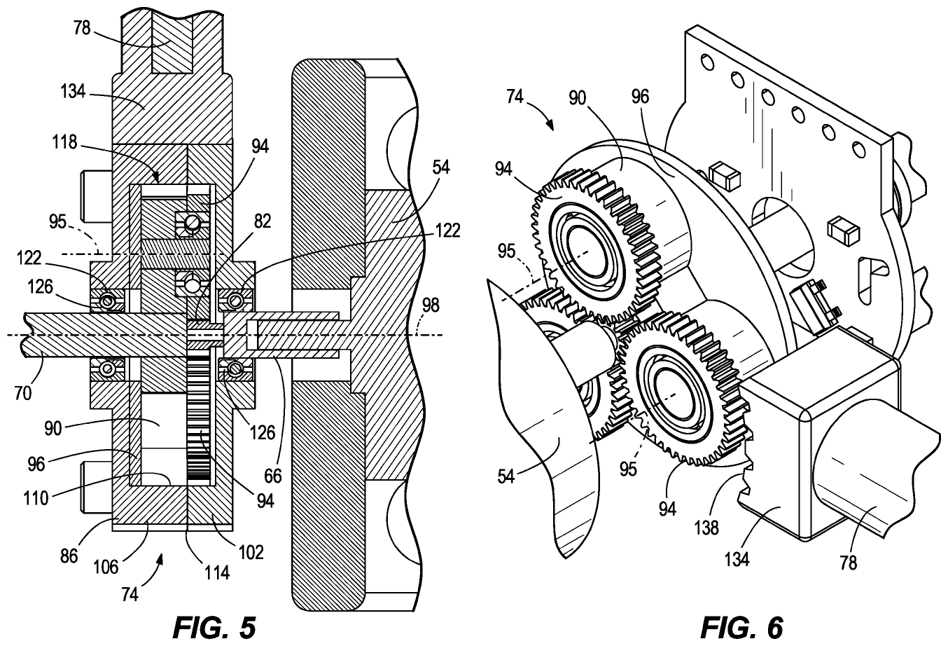

With reference to FIGS. 5 and 6, the gear set 74 includes a sun gear 82, a ring gear 86, a carrier 90, a plurality of planet gears 94, and a friction pad 96. The sun gear 82 is coupled to the input shaft 66 for co-rotation with the input shaft 66. In other words, the sun gear 82 rotates with and at the same rotational speed as the input shaft 66. In some embodiments, the input shaft 66 is the electric motor output shaft. In the illustrated embodiment, the input shaft 66 and the sun gear 82 define a central rotational axis 98.

With continued reference to FIGS. 5 and 6, the ring gear 86 includes a first portion 102 and a second portion 106 coupled to the first portion 102 with, for example, an adhesive or fastener. In some embodiments, a portion of the ring gear 86 is a cover. The ring gear 86 includes an inner circumferential surface 110 and an outer circumferential surface 114. In the illustrated embodiment, the inner circumferential surface 110 has a plurality of teeth formed thereon, and the outer circumferential surface 114 has a plurality of teeth formed thereon. In other words, the ring gear 86 in the illustrated embodiment teeth has on the outer surface 114 (e.g., to engage with the clutch 78) and teeth on the inner surface 110 (e.g., to engage with the planet gears 94). In some embodiments, the inner surface 110 of the ring gear 86 has teeth formed on the first portion 102, but not the second portion 106. In other words, the inner surface of the first portion 102 is aligned with the planet gears 94 and has teeth formed thereon, whereas the inner surface of the second portion 102 is aligned with the carrier 90 and does not have teeth formed thereon.

With reference to FIGS. 4 and 5, a cavity 118 is at least partially defined by the first portion 102 and the second portion 106. The planet gears 94, the friction pad 96, and the carrier 90 are positioned within the cavity 118. In the illustrated embodiment, the first portion 102 and the second portion 106 of the ring gear 86 each include a bore 122 to receive a bearing 126 (e.g., a roller bearing). The bearings 126 receive the input shaft 66 and the output shaft 70.

With continued reference to FIGS. 5 and 6, the plurality of planet gears 94 is coupled to the carrier 90. The carrier 90 is rotatable about the axis 98 and each of the planet gears 94 is rotatable about their own respective planet gear axis 95. The planet gears 94 are enmeshed with the teeth of the inner surface 110 on the ring gear 86. In the illustrated embodiment, the output shaft 70 is coupled to the carrier 90 for co-rotation with the carrier 90.

With reference to FIGS. 3 and 4, the clutch 78 is engageable with the outer surface 114 of the ring gear 86. In the illustrated embodiment, the clutch 78 includes a linear actuator 130 (e.g., an electric actuator) and a block 134 attached to the linear actuator 130. The block 134 includes teeth 138 that are selectively engaged with the teeth on the outer surface 114 of the ring gear 86. With the block 134 of the clutch 78 engaged with the ring gear 86, the ring gear 86 is fixed and prevented from rotating in the illustrated embodiment. In other words, the block 134 is selectively engaged and disengaged with this ring gear 86 in response to activation of the linear actuator 130. In other embodiments, the clutch includes an electrostatic clutch.

With reference to FIGS. 5 and 6, the friction pad 96 is positioned in the cavity 118. In the illustrated embodiment, the friction pad 96 is positioned between the carrier 90 and the ring gear 86. Specifically, the friction pad 96 in the illustrated embodiment is positioned axially between the carrier 90 and the second portion 106 of the ring gear 86. In the illustrated embodiment, the friction pad 96 prevents the carrier 90 from rotating relative to the ring gear 86 unless the torque applied to the sun gear 82 is above a threshold torque. In other words, the friction pad 96 presses against the carrier 90 and the ring gear 86 to prevent relative rotation unless sufficient torque is applied to the carrier 90 by the sun gear 82. In some embodiments, the friction pad 96 is made of rubber, foam, graphite, or any other suitable material.

As explained in greater detail herein, the transmission assembly 58 is movable between a first configuration and a second configuration. In the illustrated embodiment, selective engagement of the clutch 78 moves the transmission assembly 58 between the first and second configurations.

In the first configuration, the clutch 78 engages the outer surface 114 of the ring gear 86 and the transmission assembly 58 has a first gear ratio equal to N:1, where N is greater than zero (0) and not equal to one (1). In some embodiments, N is less than 1. In some embodiments, N is greater than 1. As used herein, the first gear ratio is the ratio of the input shaft 66 rotational speed to the output shaft 70 rotational speed of the gear set 74 in the first configuration. When in the first configuration, the planet gears 94 rotate about their respective planet gear axes 95 relative to the friction pad 96. More specifically, when operated in the N: 1 configuration (e.g., the first configuration), the ring gear 86 is prevented from rotating by the engaged clutch 78, and the friction pad 96 is tuned such that the planet gears 94 cannot rotate without the input torque to the gear set being greater than a minimum torque (Tmin), which is a fraction of the input actuator or mechanism maximum torque output (e.g., the maximum torque output of the motor 54).

Once a torque greater than Tmin is applied (Tapp), for example by the motor 54, the sun gear 82 drives the planet gears 94 as they overcome the friction generated by the friction pad 96. The applied torque amount above Tmin is amplified by the nominal gear ratio of the planetary gearbox, N, to provide an output torque equal to (Tapp−Tmin)*N. In other words, in the first configuration, friction caused by friction pad 96 reduces the output efficiency of the gear set 74. As such, by appropriately tuning the gearbox's nominal gear ratio N and adjusting Tmin, the maximum output torque is adjusted. Conventional gear set designs aim to reduce or remove friction from the assembly to improve efficiency and overall performance. However, the gear set 74 detailed herein adds or integrates friction (e.g., via the friction pads 96 or other frictional feature) to achieve additional functionality that surpasses any reduction in efficiency in the first configuration.

In the second configuration, the clutch 78 is disengaged from (e.g., spaced from) the outer surface 114 of the ring gear 86 and the transmission assembly 58 has a second gear ratio equal to 1:1 (e.g., a direct drive). As used herein, the second gear ratio is the ratio of the input shaft 66 rotational speed to the output shaft 70 rotational speed of the gear set 74 in the second configuration. When in the second configuration, the ring gear 86 is not prevented from rotating by the clutch 78 (e.g., the clutch 78 is disengaged), but the friction pad 96 prevents the planet gears 94 from rotating about their respective planet gear axes 95. When in the second configuration, the ring gear 86 co-rotates with the output shaft 70. Thus, the entire gear set 74 acts as a single rotating rigid body, with each component (including the output) rotating at the same speed as the input about the axis 98. In other words, the entire gear set 74 is rotating as a unitary body at the rotational speed of the input shaft 66. The gear set 74 is therefore acting as a 1:1 gearbox in the second configuration.

The gear set 74 disclosed herein provides a 2-speed active gearbox to augment, in some examples, extrinsically powered prosthetic hands. The gear set 74 automatically changes between two discrete gear ratios (e.g., 2-speed): 1:1 and N:1, where N is greater than 0 but not equal to 1. In some embodiments, N is a negative value less than 1 (e.g., the output rotational direction is opposite the input rotational direction). The output of the gear set 74 can be used to drive further transmission elements, including additional gearboxes, linkages, or lead-or ball-screws, cables, or other suitable components. Advantageously, the gear set 74 provides multi-use, multi-speed capability.

In some embodiments, the transmission assembly includes a multi-stage planetary gearbox. In such an embodiment, each stage (or at least more than one stage) has its own integrated friction pad and clutch. In other words, including at least 2 stages with friction pads in a multi-stage gear box). This enables a larger number of ratios that the gear assembly can toggle between. In some embodiments, the transmission assembly includes a first two-speed gear set and a second two-speed gear set coupled to the first two-speed gear set.

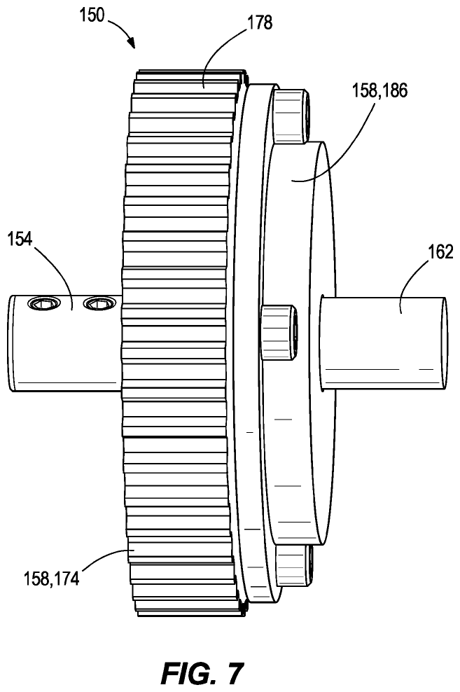

In some embodiments, the friction pad 96 is one of a plurality of friction pads integrated into the gear set. In some embodiments, the friction pad 96 is located in the gear set at a variety of positions. For example, in some embodiments, the friction pad is positioned between the planet gear and the carrier (FIGS. 7-9). In another embodiment, the friction pad is positioned between the planet gear and the ring gear. The friction pad or plurality of friction pads force two components of the gear set to rotate about the central rotational axis when the ring gear is not grounded, thereby forcing the entire gear set to rotate about the axis and creating the 1:1 direct drive.

In some embodiments, the friction pad in the gear set is replaced with an alternative frictional feature. In some embodiments, the gear set includes a non-actuated or passive frictional feature that causes the gear set to seize and behave like a single rigid body (e.g., direct drive) when an input torque applied is below a threshold. When the input torque exceeds the threshold torque, the gear set operates as it would normally with a gear ratio. In some embodiments, the frictional feature is an interference fit. For example, an interference fit between planet gears with a shaft connecting them to a carrier. As another example, an interference fit may be positioned between a carrier output shaft with a ring gear structure. In some embodiments, the frictional feature is a modification to the shape of at least one of the gears. For example, the modification may be a protrusion or groove on the sun, planet, and/or ring gear teeth such that a known load must be applied to elastically deform the gear teeth to cause the teeth to move relative to each other.

In one embodiment, a gear set includes an input shaft; an output shaft; and a plurality of gears including at least one frictional feature that causes the plurality of gears to rotate in unison when an input torque on the input shaft is below a threshold torque. The gear set has a gear ratio for the input shaft rotational speed to the output shaft rotational speed equal to 1:1 when the input torque is below the threshold torque. In some embodiments, the at least one frictional feature is a friction pad. In other embodiments, the at least one frictional feature is an interference fit, a protrusion, a friction pad, or a combination thereof.

With reference to FIG. 7-9, a gear set 150 includes an input sun gear 154, a ring gear 158, an output carrier 162, a plurality of planet gears 166, and a plurality of friction pads 170.

The ring gear 158 includes a first portion 174 with teeth formed on an outer circumferential surface 178 and teeth formed on an inner circumferential surface 182. The ring gear 158 also includes a second portion (e.g., a rear cover) 186.

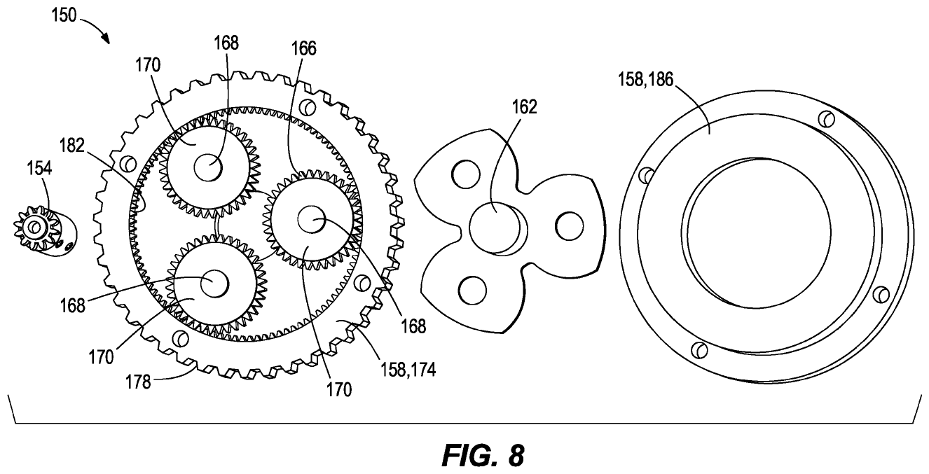

With reference to FIG. 8, each of the plurality of friction pads 170 is positioned between the planet gears 166 and the output carrier 162. In the illustrated embodiment, three ring-shaped friction pads 170 are secured to an axial end face of each of the planet gears 166. As such, the friction pads 170 are aligned with a rotational axis 168 of each of the planet gears 166.

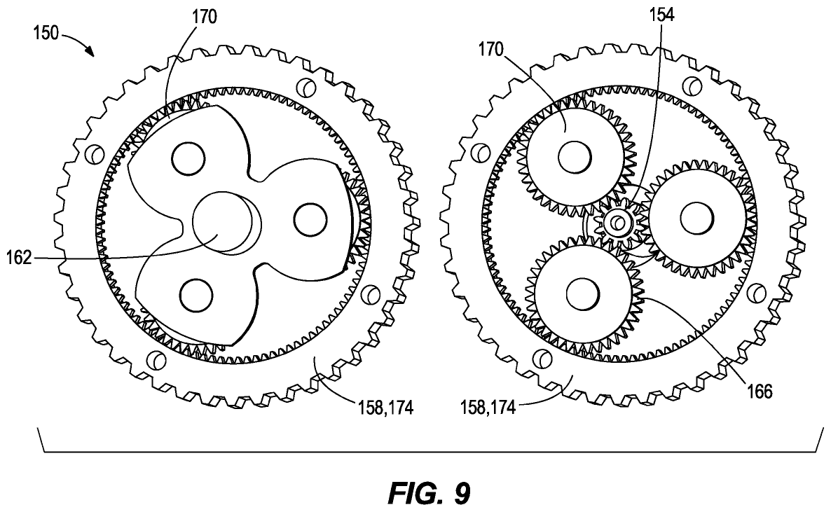

With reference to FIG. 9, the gear set 150 is assembled with the friction pads 170 positioned axially between the planet gears 166 and the carrier 162. In the illustrated embodiment, the sun gear 154 is enmeshed with three planet gears 166, and the planet gears 166 are enmeshed with teeth on the inner surface 182 of the ring gear 158. As explained with respect to the gearset of FIG. 2, a clutch (e.g., the clutch 78) selectively engages the outer surface 178 of the ring gear 158 to switch the gear set 150 between two configurations (e.g., a 1:1 gear ratio configuration and a N:1 gear ratio configuration).

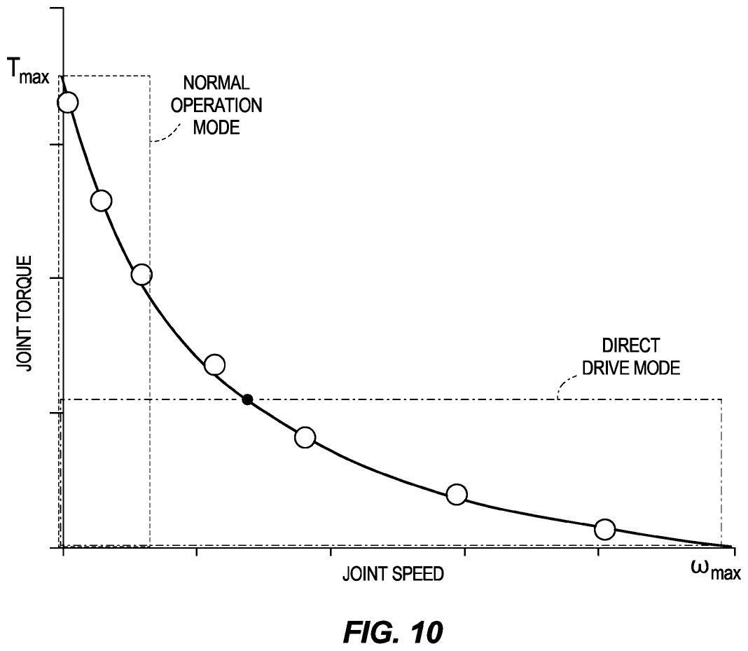

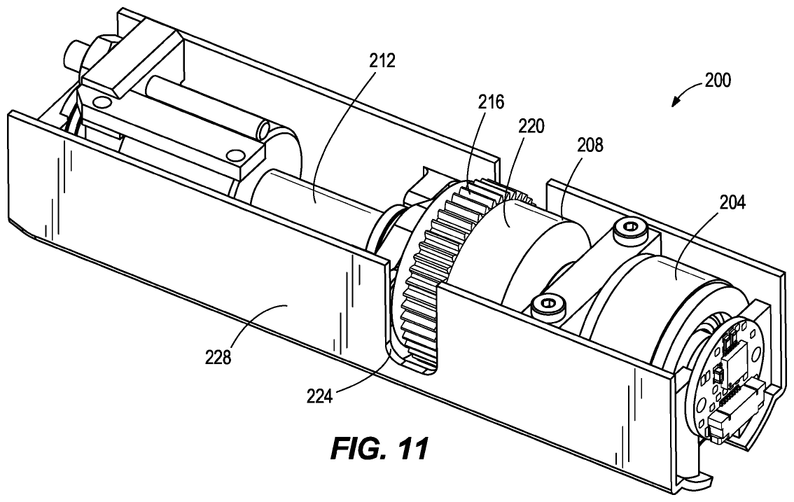

With reference to FIG. 10, a graph of joint torque with respect to joint speed illustrates the advantage of the gear sets detailed herein. To replicate human joint kinematics, the assembly requires large torque with low speeds, and high speeds with low torque. This capability is provided by the gear sets herein by switching between the two configurations. A “normal” operation mode (e.g., a N:1 gear ratio) is now theoretically less efficient because of the integration of at least one friction pad into the gear set. When the clutch is released, a direct drive configuration (e.g., a 1:1 gear ratio) is provided for the high speed, low torque application. In the direct drive configuration, there is no theoretical efficiency loss from the friction pad(s), because there is no movement relative to the friction pad in the gear set.

In some embodiments, a gear set includes an input shaft, an output shaft, a gear, and at least one friction pad. The gear set operates in a first configuration with a first gear ratio for the input shaft rotational speed to the output shaft rotational speed equal to N:1, where N is not equal to 1. In some embodiments, N is a negative value less than 0. In the first configuration, the gear rotates relative to the friction pad. The gear set also operates in a second configuration with a second gear ratio for the input shaft rotational speed to the output shaft rotational speed equal to 1:1. In the second configuration, the friction pad prevents rotation of the gear relative to the friction pad. In some embodiments, the gear is a planet gear of a planetary gear set. In other embodiments, the gear is a ring gear of a planetary gear set.

With reference to FIG. 11, an assembly 200 includes an electric motor 204, a gear set 208, and a linkage 212 coupled to the gear set 208. As disclosed herein, a prosthetic, for example, may be coupled to the linkage 212. In a first configuration, the gear set 208 has a first gear ratio of N:1, where N is not equal to 1. In a second configuration, the gear set 208 has a gear ratio of 1:1. In the illustrated embodiment, the gear set 208 is a cycloidal gear set. In the illustrated embodiment, the gear set 208 includes a plurality of teeth 216 formed on an outer surface 220. As detailed herein, the plurality of teeth 216 may be engaged by an actuator to switch the gear set 208 between two configurations. In the illustrated embodiment, at least some of the teeth 216 extend through an opening 224 (e.g., a notch, an aperture) formed in a housing 228.

One skilled in the art will readily appreciate that the present disclosure is well adapted to carry out the objects and obtain the ends and advantages mentioned, as well as those inherent herein. The present disclosure described herein are exemplary embodiments and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Various features and advantages are set forth in the following claims.

What is claimed is:

1. An assembly comprising:
an input shaft;
a sun gear coupled to the input shaft for co-rotation with the input shaft;
a ring gear with an inner surface and an outer surface;
a carrier;
a planet gear coupled to the carrier and enmeshed with the inner surface of the ring gear;
an output shaft coupled to the carrier for co-rotation with the carrier;
a friction pad positioned between the planet gear and the carrier, or positioned between the carrier and the ring gear, or positioned between the planet gear and the ring gear;
a clutch engageable with the outer surface of the ring gear;
wherein the assembly is movable between a first configuration and a second configuration,
in the first configuration, the clutch engages the outer surface of the ring gear and the assembly has a first gear ratio of the input shaft rotational speed to the output shaft rotational speed equal to N:1, where N is greater than 0 and not equal to 1; and
in the second configuration, the clutch disengages the outer surface of the ring gear and the assembly has a second gear ratio of the input shaft rotational speed to the output shaft rotational speed equal to 1:1.

2. The assembly of claim 1, wherein when in the second configuration, the ring gear co-rotates with the output shaft.

3. The assembly of claim 1, wherein the friction pad is made of rubber, foam, or graphite.

4. The assembly of claim 1, wherein when in the second configuration, the friction pad prevents the planet gear from rotating about a planet gear axis.

5. The assembly of claim 4, wherein when in the first configuration, the planet gear rotates about the planet gear axis relative to the friction pad.

6. The assembly of claim 1, wherein the ring gear includes a first portion and a second portion coupled to the first portion, wherein a cavity is at least partially defined by first portion and the second portion.

7. The assembly of claim 6, wherein the planet gear, the friction pad, and the carrier are positioned within the cavity.

8. The assembly of claim 1, wherein the clutch includes a linear actuator.

9. The assembly of claim 1, wherein the clutch includes an electrostatic clutch.

10. A gear set comprising:
an input shaft;
an output shaft;
a gear; and
a friction pad;
wherein in a first configuration the gear set has a first gear ratio for the input shaft rotational speed to the output shaft rotational speed equal to N:1, where N is not equal to 1, wherein the gear rotates relative to the friction pad in the first configuration; and
wherein in a second configuration the gear set has a gear ratio for the input shaft rotational speed to the output shaft rotational speed equal to 1:1, wherein the friction pad prevents rotation of the gear relative to the friction pad.

11. The gear set of claim 10, wherein the gear set is a cycloidal gear set.

12. The gear set of claim 10, wherein the gear set is a Ravigneaux gear set.

13. The gear set of claim 10, wherein the gear set is a planetary gear set.

14. The gear set of claim 13, wherein the gear is a planet gear.

15. The gear set of claim 13, wherein the gear is a ring gear.

16. A gear set comprising:
an input shaft;
an output shaft;
a plurality of gears including at least one frictional feature that causes the plurality of gears to rotate in unison when an input torque on the input shaft is below a threshold torque; and
wherein the gear set has a gear ratio for the input shaft rotational speed to the output shaft rotational speed equal to 1:1 when the input torque is below the threshold torque.

17. The gear set of claim 16, wherein at least one frictional feature is a friction pad.

18. The gear set of claim 16, wherein at least one frictional feature is an interference fit, a protrusion, a friction pad, or a combination thereof.

19. The gear set of claim 16, wherein the plurality of gears are part of a planetary gear set, a cycloidal gear set, or a Ravigneaux gear set.

20. An assembly comprising:
an electric motor;
a gear set including a friction pad, wherein the gear set is coupled to the electric motor;
a linkage coupled to the gear set;
a prosthetic coupled to the linkage;
wherein in a first configuration the gear set has a first gear ratio of N:1, where N is not equal to 1; and
wherein in a second configuration the gear set has a gear ratio of 1:1.

21. The assembly of claim 20, wherein the linkage includes a ball screw, a lead screw, a cable, a four-bar linkage, or a six-bar linkage.

22. The assembly of claim 20, wherein the prosthetic is a finger with a finger joint and the link is coupled between the ball screw and the finger joint.

23. The assembly claim 20, wherein the prosthetic is a finger, a thumb, a wrist, a forearm, a shoulder, a leg, a bird wing, a snake body, or a fish tail.

24. The assembly of claim 20, wherein the two-speed gear set is a first two-speed gear set and the assembly further includes a second two-speed gear set coupled to the first two-speed gear set.
